(19) United States
(12) Patent Application Publication
(10) Pub. No.: US 2003/0208242 A1
(43) Pub. Date: Nov. 6, 2003

(54) Electropancreatography

(76) Inventors: Tamar Harel, Haifa (IL); Bella Felsen, Haifa (IL); Tamir Levi, Haifa (IL); Yuval Mika, Shmurat Zichron (IL); Offer Glassberg, Haifa (IL); Nissim Darvish, Zrufa (IL)

Correspondence Address: William H Dippert, Reed Smith, 29th Floor, 599 Lexington Avenue, New York, NY, 10022, US

(21) Appl. No.: 10/296,668

(22) PCT Filed: May 30, 2001
(86) PCT No.: PCT/IL01/00501

(51) Int. Cl. A61N 1/18

(52) U.S. Cl. 607/40

(57) ABSTRACT

Apparatus (18) for sensing electrical activity of a pancreas (20) of a patient is provided. The apparatus includes a set of one or more electrodes (100), adapted to be coupled to the pancreas, and a control unit (90), adapted to receive electrical signals from the electrodes indicative of electrical activity of pancreatic cells which are in a plurality of islets of the pancreas, and to generate an output responsive thereto.

### FIELD OF THE INVENTION

The present invention relates generally to electrical sensing, and specifically to invasive devices and methods for sensing electrical activity of the pancreas.

### BACKGROUND OF THE INVENTION

The human pancreas performs two functions: producing pancreatic endocrine hormones, which affect the behavior of cells throughout the body, and producing pancreatic digestive enzymes, which assist in the digestion of food. Among other endocrine hormones produced by the pancreas, insulin is the most well-known, because of the large number of diabetic patients who regularly monitor their glucose levels to determine whether to self-administer a dose of insulin. The general function of insulin is to regulate blood glucose levels, by causing peripheral cells of the body to absorb glucose as a person's blood sugar rises. Some types of diabetes, for example, arise as a consequence of inadequate insulin release by the pancreas. Normal, physiological insulin generation and uptake, however, allow peripheral cells to properly manage the body's energy needs.

It is well known in the art to measure the electrical activity of individual pancreatic beta cells, for example, by micropipetting. It is also known to measure the collective activity of the cluster of cells in a pancreatic islet of Langerhans.

An article by Jaremko and Rorstad, entitled, “Advances toward the implantable artificial pancreas for treatment of diabetes,” Diabetes Care, 21(3), March 1998, which is incorporated herein by reference, describes enzymatic glucose sensors and optical glucose sensors for use in an artificial pancreas. They note that “. . . implantable enzymatic sensors are not yet clinically applicable because of problems with bio-compatibility. Clinical research is necessary on the effect of chronic subcutaneous implantation and local inflammation on glucose sensor performance.” Moreover, with respect to optical sensors, they write: “It appears that despite recent press releases, we are still some way from having a widely applicable long-term optical blood glucose sensor. This technology avoids the biocompatibility problems of enzymatic sensors but improvements in precision and reductions in cost are needed. Basic research is required as to the effects of environmental and metabolic variations on absorption spectra before a reliable and clinically practical optical sensor will become available.” They similarly describe subcutaneous microdialysis probes and a transcutaneous glucose extraction device as not yet being suitable for regular clinical use. They conclude, “the quest for a reliable, long-term, wearable, or implantable blood glucose sensor has been frustrating so far and few clinical studies have been carried out.”

U.S. Pat. No. 6,093,167 to Houben et al., which is incorporated herein by reference, describes implantable apparatus for monitoring pancreatic beta cell electrical activity in a patient in order to obtain a measure of the patient's insulin demand and blood glucose level. A stimulus generator delivers stimulus pulses, which are intended to synchronize pancreatic beta cell depolarization and to thereby produce an electrical response in the pancreas. This response is analyzed so as to determine an indication of insulin demand, whereupon insulin from an implanted pump is released, or the pancreas is stimulated so as to enhance insulin production.

U.S. Pat. No. 5,919,216 to Houben et al., which is incorporated herein by reference, describes a system for automatically responding to insulin demand without any need for external monitoring or injecting of insulin into a diabetic patient. The system as described senses glucose levels internally, and responds by stimulating either the pancreas or a transplant of pancreatic islets in order to enhance insulin production.

U.S. Pat. No. 5,741,211 to Renirie et al., which is incorporated herein by reference, describes apparatus which evaluates an electrocardiographic signal in order to determine an indication of blood insulin and/or glucose levels.

U.S. Pat. Nos. 5,101,814 and 5,190,041 to Palti, which are incorporated herein by reference, describe a system which utilizes implanted glucose-sensitive living cells to monitor blood glucose levels. The implanted cells produce a detectable electrical or optical signal in response to changes in glucose concentration in surrounding tissue. The signal is then detected and interpreted to give a reading indicative of blood glucose levels.

The following articles, which are incorporated herein by reference, may be of interest. In particular, methods and apparatus described in one or more of these articles may be adapted for use with some preferred embodiments of the present invention.

1) Lamb F. S. et al., “Cyclosporine augments reactivity of isolated blood vessels,” Life Sciences, 40, pp. 2571-2578, 1987.

2) Johansson B. et al., “Static and dynamic components in the vascular myogenic response to passive changes in length as revealed by electrical and mechanical recordings from the rat portal vein,” Circulation Research, 36, pp. 76-83, 1975.

3) Zelcer E. et al., “Spontaneous electrical activity in pressurized small mesenteric arteries,” Blood Vessels, 19, pp. 301-310, 1982.

4) Schobel H. P. et al., “Preeclampsia—a state of sympathetic overactivity,” New England Journal of Medicine, 335, pp. 1480-1485, 1996.

5) Gomis A. et al., “Oscillatory patterns of electrical activity in mouse pancreatic islets of Langerhans recorded in vivo,” Pflugers Archiv European Journal of Physiology, Abstract Volume 432(3), pp. 510-515, 1996.

6) Soria B. et al., “Cytosolic calcium oscillations and insulin release in pancreatic islets of Langerhans,” Diabetes Metab., 24(1), pp. 37-40, February 1998.

7) Magnus G. et al., “Model of beta-cell mitochondrial calcium handling and electrical activity. II. Mitochondrial variables,” American Journal of Physiology, 274(4 Pt 1): C1174-1184, April 1998.

8) Gut R. et al., “High-precision EMG signal decomposition using communication techniques,” IEEE Transactions on Signal Processing, 48(9), pp. 2487-2494, September 2000.

9) Nadal A. et al., “Homologous and heterologous asynchronicity between identified alpha-, beta-, and delta-cells within intact islets of Langerhans in the mouse,” Journal of Physiology, 517(Pt. 1), pp. 85-93, May 1999.

### SUMMARY OF THE INVENTION

It is an object of some aspects of the present invention to provide improved methods and apparatus for sensing pancreatic electrical activity.

It is also an object of some aspects of the present invention to provide methods and apparatus for sensing electrical activity of a substantial portion of the pancreas.

It is a further object of some aspects of the present invention to provide improved methods and apparatus for modifying pancreatic function.

It is yet a further object of some aspects of the present invention to provide improved methods and apparatus for treating physiological disorders resulting from improper functioning of the pancreas.

It is still a further object of some aspects of the present invention to provide improved methods and apparatus for monitoring glucose levels in the blood.

In preferred embodiments of the present invention, pancreatic apparatus comprises a control unit and one or more electrodes, adapted to be coupled to respective sites on, in, or near the pancreas of a human subject. Preferably, the electrodes convey to the control unit electrical signals which are generated within a substantial portion of the pancreas. Typically, but not necessarily, the control unit analyzes various aspects of the signals, and drives the electrodes to apply pancreatic control signals to the pancreas responsive to the analysis. The term “substantial portion of the pancreas,” as used in the context of the present patent application and in the claims, is to be understood as a portion of the pancreas larger than two or more islets, and typically larger than ten or more islets.

These embodiments of the present invention thus differ significantly from prior art electrical sensors, which typically measure the behavior of (a) individual pancreatic cells, or (b) clusters of cells in a particular islet. The inventors believe that neither of these prior art methods is appropriate for daily use by patients. The former is typically practiced using a micropipette, which cannot be used practically by a patient (because of the likelihood of the micropipette breaking and/or the damage to a pancreatic cell due to the micropipetting), and the latter reveals the electrical activity of only a very small portion of the pancreas. Thus, the prior art measures islet activity by actually placing a probe in contact with the islet, and, typically, applying suction to the islet. These embodiments of the present invention, by contrast, do not necessarily require contact of an electrode or other probe with an islet; indeed, a single electrode often records activity in a plurality of islets.

By way of analogy, the behavior of the heart cannot be adequately summarized by assessing the electrical activity of any one bundle of cells; instead, an electrocardiogram is used. Embodiments of the present invention, similarly, assess the electrical activity of a substantial portion of the pancreas, typically in order to determine whether a treatment is appropriate (e.g., stimulating the pancreas to secrete more insulin, or generating a signal to activate an implanted insulin pump). For this reason, the inventors call the process of sensing the electrical activity of a substantial portion of the pancreas, as described herein, electropancreatography (EPG). Experiments performed by the inventors have shown that electropancreatography is sensitive to clinically-significant phenomena, e.g., an increase in blood glucose levels from normal to supraphysiological values.

In a preferred embodiment, the control unit drives some or all of the electrodes to apply signals to the pancreas responsive to detecting EPG signals which are indicative of a particular physiological condition, such as elevated blood glucose levels. Preferably, these signals are applied using methods and apparatus similar to those described in one or more of the following applications: (a) U.S. Provisional Patent Application No. 60/123,532, filed Mar. 5, 1999, entitled “Modulation of insulin secretion,” (b) PCT Patent Application IL 00/00132, filed Mar. 5, 2000, entitled “Blood glucose level control,” or (c) PCT Patent Application IL 00/00566, filed Sep. 13, 2000, also entitled “Blood glucose level control.” Each of these applications is assigned to the assignee of the present patent application and is incorporated herein by reference. Typically, each electrode conveys a particular waveform to the pancreas, which may differ in certain aspects from the waveforms applied to other electrodes. The particular waveform to be applied to each electrode is preferably determined by the control unit, initially under the control of a physician during a calibration period of the unit. After the initial calibration period, the unit is generally able to automatically modify the waveforms as needed to maintain a desired level of performance of the apparatus.

In a preferred embodiment, one or more physiological sensors (e.g., for measuring blood sugar, blood pH, pCO2, pO2, blood insulin levels, blood ketone levels, ketone levels in expired air, blood pressure, respiration rate, respiration depth, or heart rate) send physiological-sensor signals to the control unit. The various sensor signals serve as feedback, to enable the control unit to iteratively adjust the signals applied to the pancreas. Alternatively or additionally, other sensors are coupled to the pancreas or elsewhere on the patient's body, and send signals to the control unit which are used in determining modifications to parameters of the applied signals.

As appropriate, methods and apparatus described in U.S. Provisional Patent Application No. 60/208,157, entitled, “Electrical Activity Sensor for the Whole Pancreas,” filed May 31, 2000, which is assigned to the assignee of the present patent application and is incorporated herein by reference, may be adapted for use with embodiments of the present invention.

There is therefore provided, in accordance with a preferred embodiment of the present invention, apparatus for sensing electrical activity of a pancreas of a patient, including:

a set of one or more electrodes, adapted to be coupled to the pancreas; and

a control unit, adapted to receive electrical signals from the electrodes indicative of electrical activity of pancreatic cells which are in a plurality of islets of the pancreas, and to generate an output responsive thereto.

Preferably, a single electrode in the set of one or more electrodes is adapted to convey to the control unit an electrical signal indicative of electrical activity of pancreatic cells which are in two or more of the islets.

In a preferred embodiment, the control unit is adapted to receive electrical signals from the electrodes indicative of electrical activity of pancreatic cells which are in five or more of the islets. Preferably, the control unit is adapted to receive electrical signals from the electrodes indicative of electrical activity of pancreatic cells which are in ten or more of the islets.

In a preferred embodiment, a first one of the one or more electrodes is adapted to convey to the control unit a first electrical signal, indicative of electrical activity of pancreatic cells which are in a first one of the islets, and wherein a second one of the one or more electrodes is adapted to convey to the control unit a second electrical signal, indicative of electrical activity of pancreatic cells which are in a second one of the islets, which is different from the first one of the islets.

The control unit is typically adapted to receive the electrical signals from the electrodes responsive to spontaneous electrical activity of the pancreatic cells.

For some applications, the control unit is adapted to analyze the signals so as to identify an aspect thereof indicative of activity of a type of cell selected from the list consisting of: pancreatic alpha cells, pancreatic beta cells, pancreatic delta cells, and polypeptide cells, and wherein the control unit is adapted to generate the output responsive to identifying the aspect.

There is further provided, in accordance with a preferred embodiment of the present invention, apparatus for monitoring a glucose level of a patient, including:

a set of one or more electrodes, adapted to be coupled to a pancreas of the patient; and

a control unit, adapted to receive electrical signals from the electrodes indicative of spontaneous electrical activity of pancreatic cells, to analyze the signals so as to determine a change in the glucose level, and to generate an output responsive to determining the change.

There is still further provided, in accordance with a preferred embodiment of the present invention, apparatus for analyzing electrical activity of a pancreas of a patient, including:

a set of one or more electrodes, adapted to be coupled to the pancreas; and

a control unit, adapted to receive electrical signals from the electrodes, adapted to analyze the signals so as to identify an aspect thereof which is indicative of activity of pancreatic alpha cells, and adapted to generate an output responsive to identifying the aspect.

There is yet further provided, in accordance with a preferred embodiment of the present invention, apparatus for analyzing electrical activity of a pancreas of a patient, including:

a set of one or more electrodes, adapted to be coupled to the pancreas; and

a control unit, adapted to receive electrical signals from the electrodes, adapted to analyze the signals so as to identify an aspect thereof which is indicative of spontaneous activity of pancreatic beta cells, and adapted to generate an output responsive to identifying the aspect.

Typically, the control unit is adapted to analyze the signals so as to distinguish between the aspect thereof which is indicative of the activity of the beta cells and an aspect thereof which is indicative of activity of pancreatic alpha cells, and wherein the control unit is adapted to generate the output responsive to distinguishing between the aspects.

There is also provided, in accordance with a preferred embodiment of the present invention, apparatus for analyzing electrical activity of a pancreas of a patient, including:

a set of one or more electrodes, adapted to be coupled to the pancreas; and

a control unit, adapted to receive electrical signals from the electrodes, adapted to analyze the signals so as to identify an aspect thereof which is indicative of activity of pancreatic delta cells, and adapted to generate an output responsive to identifying the aspect.

There is additionally provided, in accordance with a preferred embodiment of the present invention, apparatus for analyzing electrical activity of a pancreas of a patient, including:

a set of one or more electrodes, adapted to be coupled to the pancreas; and

a control unit, adapted to receive electrical signals from the electrodes, adapted to analyze the signals so as to identify an aspect thereof which is indicative of activity of polypeptide cells, and adapted to generate an output responsive to identifying the aspect.

In a preferred embodiment, the control unit is adapted to compare the aspect of the signals with a stored pattern that is indicative of activity of the cells, and to generate the output responsive thereto.

For some applications, the control unit is adapted to analyze the signals by means of a technique selected from the list consisting of: single value decomposition and principle component analysis, and to generate the output responsive thereto. Alternatively, the control unit is adapted to analyze the signals under an assumption that the activity of the cells is dependent on electrical activity of another type of pancreatic cell, and to generate the output responsive thereto.

Typically, the control unit is adapted to analyze the signals so as to identify a frequency aspect thereof, and to generate the output responsive to identifying the frequency aspect. For example, the control unit may be adapted to analyze the signals so as to differentiate between a first frequency aspect of the signals which is indicative of the activity of the cells, and a second frequency aspect of the signals, different from the first frequency aspect, which is indicative of activity of another type of pancreatic cell.

For some applications, the control unit is adapted to analyze the signals so as to identify over time a change in the frequency aspect that is characteristic of the cells.

Alternatively or additionally, the control unit is adapted to analyze the signals so as to identify a magnitude aspect thereof, wherein the control unit is adapted to analyze the frequency aspect and the magnitude aspect in combination, and wherein the control unit is adapted to generate the output responsive to analyzing the aspects. Further alternatively or additionally, the control unit is adapted to analyze the signals so as to identify a duration aspect thereof, wherein the control unit is adapted to analyze the frequency aspect and the duration aspect in combination, and wherein the control unit is adapted to generate the output responsive to analyzing the aspects.

For some applications, the control unit is adapted to analyze the signals so as to identify a magnitude aspect thereof and a duration aspect thereof, the control unit is adapted to analyze the aspects in combination, and the control unit is adapted to generate the output responsive to analyzing the aspects.

The control unit is preferably adapted to analyze the electrical signals with respect to calibration data indicative of aspects of pancreatic electrical activity recorded at respective times, in which respective measurements of blood glucose level of the patient generated respective values.

Preferably, the electrodes are adapted to be placed in contact with the pancreas, e.g., in contact with the head, body, or tail of the pancreas. Alternatively or additionally, at least one of the electrodes is adapted to be placed in contact with a vein or artery of the pancreas, or in contact with a blood vessel in a vicinity of the pancreas.

For some applications, the apparatus includes a treatment unit, adapted to receive the output and to apply a treatment to the patient responsive to receiving the output. The treatment unit preferably includes at least one electrode in the set of electrodes, and the control unit is adapted to drive the at least one electrode to apply current to the pancreas capable of treating a condition of the patient. Alternatively or additionally, the treatment unit includes a signal-application electrode, not necessarily in the set of electrodes, and the control unit is adapted to drive the signal-application electrode to apply current to the pancreas capable of treating a condition of the patient.

In a preferred embodiment, the control unit is adapted to generate the output responsive to an aspect of the timing of the electrical signals, and the treatment unit is adapted to apply the treatment responsive to the timing aspect.

The control unit is typically adapted to configure the output to the treatment unit so as to be capable of modifying an amount of glucose in blood in the patient, e.g., so as to be capable of increasing or decreasing the amount of glucose.

In a preferred embodiment, the control unit is adapted to receive the signals from at least one of the electrodes when the at least one of the electrodes is not in contact with any islet of the pancreas.

For some applications, the control unit is adapted to generate the output so as to facilitate an evaluation of a state of the patient, not necessarily in conjunction with any treatment of the patient.

In a preferred embodiment, at least one of the electrodes has a characteristic diameter less than about 3 millimeters. For example, the at least one of the electrodes may have a characteristic diameter less than about 300 microns. For some applications, the at least one of the electrodes has a characteristic diameter less than about 30 microns.

In a preferred embodiment, the apparatus includes a clip mount, coupled to at least one of the electrodes, which is adapted for securing the at least one of the electrodes to the pancreas.

There is yet additionally provided, in accordance with a preferred embodiment of the present invention, a method for sensing electrical activity of a pancreas of a patient, including:

receiving electrical signals indicative of electrical activity of pancreatic cells which are in a plurality of islets of the pancreas; and

generating an output responsive thereto.

There is still additionally provided, in accordance with a preferred embodiment of the present invention, a method for monitoring a glucose level of a patient, including:

receiving electrical signals indicative of spontaneous electrical activity of pancreatic cells;

analyzing the signals so as to determine a change in the glucose level; and

generating an output responsive to determining the change.

There is also provided, in accordance with a preferred embodiment of the present invention, a method for analyzing electrical activity of a pancreas of a patient, including:

receiving electrical signals recorded at one or more pancreatic sites;

analyzing the signals so as to identify an aspect thereof which is indicative of activity of pancreatic alpha cells; and

generating an output responsive to identifying the aspect.

There is further provided, in accordance with a preferred embodiment of the present invention, a method for analyzing electrical activity of a pancreas of a patient, including:

receiving electrical signals recorded at one or more pancreatic sites;

analyzing the signals so as to identify an aspect thereof which is indicative of activity of pancreatic beta cells; and

generating an output responsive to identifying the aspect.

There is still further provided, in accordance with a preferred embodiment of the present invention, a method for analyzing electrical activity of a pancreas of a patient, including:

receiving electrical signals recorded at one or more pancreatic sites;

analyzing the signals so as to identify an aspect thereof which is indicative of activity of pancreatic delta cells; and

generating an output responsive to identifying the aspect.

There is yet further provided, in accordance with a preferred embodiment of the present invention, a method for analyzing electrical activity of a pancreas of a patient, including:

receiving electrical signals recorded at one or more pancreatic sites;

analyzing the signals so as to identify an aspect thereof which is indicative of activity of polypeptide cells; and

generating an output responsive to identifying the aspect.

The present invention will be more fully understood from the following detailed description of the preferred embodiments thereof, taken together with the drawings, in which:

### BRIEF DESCRIPTION OF THE DRAWINGS

FIG. 1 is a schematic illustration of the external surface of a pancreas, showing the placement of electrodes thereon, in accordance with a preferred embodiment of the present invention;

FIGS. 2, 3A and 3B are schematic illustrations of electrodes for sensing activity of the pancreas, in accordance with respective preferred embodiments of the present invention;

FIG. 4 is a schematic block diagram of a control unit, which receives signals from the electrodes shown in FIG. 1, in accordance with a preferred embodiment of the present invention;

FIGS. 5A, 5B, 5C, 6A, 6B, 6C, 7A, 7B, 7C, 8A, 8B, 8C, 9A, 9B, 10A, and 10B are graphs showing measurements or analysis of electrical activity taken during experiments performed in accordance with a preferred embodiment of the present invention;

FIGS. 11, 12, and 13 show the results of signal processing of the experimental results shown in FIGS. 9A and 9B, in accordance with a preferred embodiment of the present invention;

FIG. 14 shows the results of signal processing of experiments performed on dogs, in accordance with a preferred embodiment of the present invention;

FIG. 15 shows the results of electrical activity measurements made in the gastrointestinal tract and in the pancreas of a dog, during experiments performed in accordance with a preferred embodiment of the present invention;

FIG. 16 shows additional measurements of pancreatic and GI tract electrical activity, during experiments on a dog performed in accordance with a preferred embodiment of the present invention; and

FIG. 17 shows measurements of pancreatic electrical activity, during experiments on a dog performed in accordance with a preferred embodiment of the present invention.

### DETAILED DESCRIPTION OF PREFERRED EMBODIMENTS

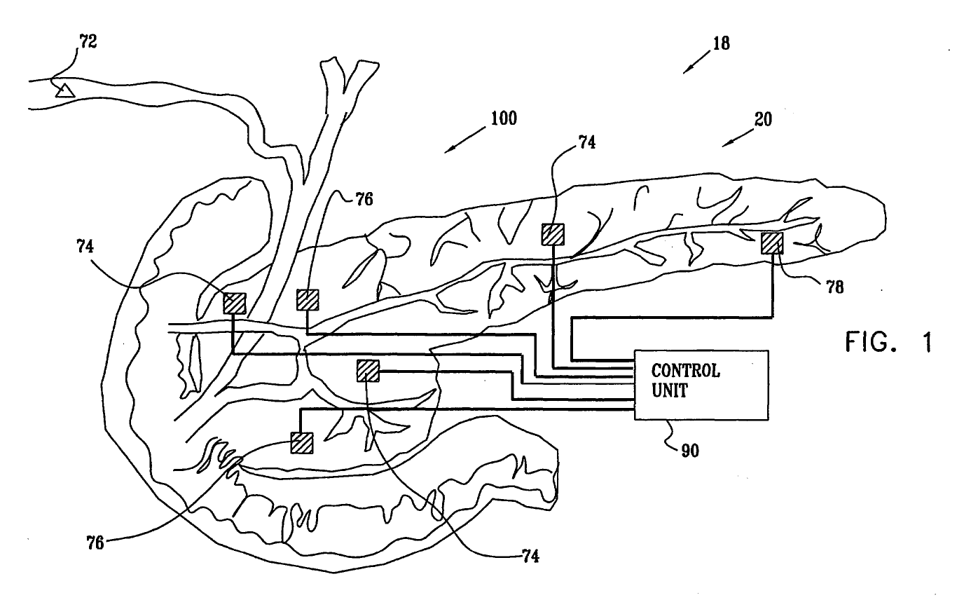

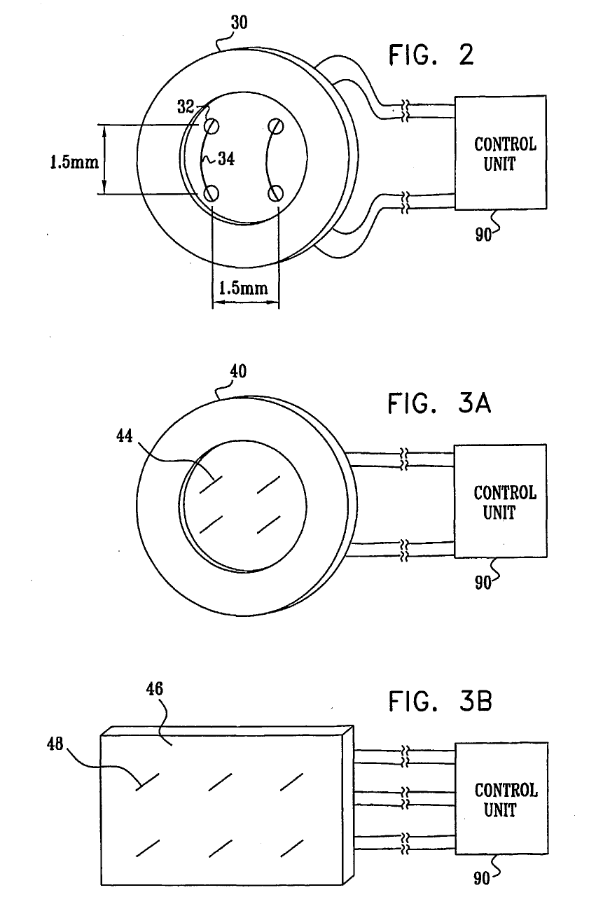

Reference is now made to FIGS. 1, 2, 3A and 3B. FIG. 1 is a schematic illustration of apparatus 18, which senses electrical activity of the pancreas 20 of a patient, in accordance with a preferred embodiment of the present invention. FIG. 2 is a schematic illustration of one portion of a clip mount 30 for application of wire electrodes 34 to the surface of pancreas 20, in accordance with a preferred embodiment of the present invention. For some applications, two or more clip mounts 30 are coupled together (e.g., by a spring or other mechanical element) to mechanically secure electrodes 34 to the pancreas. Alternatively, a one-piece clip mount having spring-like properties may be used to secure one or more electrodes to the pancreas. FIGS. 3A and 3B are schematic illustrations of respective mounts 40 and 46 for application of needle electrodes 44 and 48 to pancreas 20, in accordance with preferred embodiments of the present invention.

Apparatus 18 preferably comprises an implantable or external control unit 90, which receives signals from local sense electrodes 74 preferably located in and/or on the pancreas (e.g., near a blood vessel of the pancreas), and supplemental sensors 72 preferably located on the pancreas or elsewhere in and/or on the body of the patient. In some applications (not shown), it is desirable to insert one or more of electrodes 100 into a blood vessel in a vicinity of pancreas 20, so as to sense or stimulate from that site.

In response to receiving and analyzing the signals from local sense electrodes 74, control unit 90 may apply a treatment by means of a treatment unit, comprising, for example, one or more electrodes 100, which in turn comprise local sense electrodes 74 (FIG. 1), signal application electrodes 76 (FIG. 1), reference electrodes 78 (FIG. 1), electrodes 34 (FIG. 2), electrodes 44 (FIG. 3A) and/or electrodes 48 (FIG. 3B). Alternatively or additionally, the treatment unit may comprise other apparatus known in the art (not shown), for example, an implanted insulin pump or a display unit instructing the patient to inject a particular dose of insulin.

Typically, electrodes 100 convey signals to control unit 90 responsive to spontaneous electrical activity of the pancreas, e.g., activity which occurs in the course of natural, ongoing processes of the pancreas. For some applications, however, a synchronizing signal is first applied (e.g., using techniques described in U.S. Pat. Nos. 5,919,216 or 6,093,167 to Houben et al.), and pancreatic electrical activity is measured subsequent thereto.

In a preferred embodiment, one or more reference electrodes 78 are placed near the pancreas or elsewhere in or on the patient's body. Optionally, at least one of electrodes 78 comprises a metal case of control unit 90. It is believed that in some applications, the use of the reference electrodes minimizes problems in recording pancreatic electrical activity which may arise due to respiratory movements, neural firing, cardiac electrical phenomena, electromyographic phenomena, and/or gastrointestinal tract electrical phenomena.

For applications in which the control unit applies signals to the pancreas, methods and techniques are preferably employed which are described in one or more of the following applications: (a) U.S. Provisional Patent Application No. 60/123,532, filed Mar. 5, 1999, entitled “Modulation of insulin secretion,” (b) PCT Patent Application IL 00/00132, filed Mar. 5, 2000, entitled “Blood glucose level control,” or (c) PCT Patent Application IL 00/00566, filed Sep. 13, 2000, also entitled “Blood glucose level control.” Each of these applications is assigned to the assignee of the present patent application and is incorporated herein by reference.

Alternatively or additionally, the control unit actuates other means for responding to particular conditions detected by electrodes 100. In a preferred embodiment, an insulin pump (not shown) is actuated to deliver a determined dose of insulin to the patient. In another preferred embodiment, the control unit generates a signal which instructs the patient to self-administer a dose of insulin. In still another preferred embodiment of the present invention, apparatus 18 is used in a diagnostic mode, and electrical measurements made by the apparatus are stored for later analysis by a physician.

Preferably, control unit 90 is coupled to one or more local sense electrodes 74, which are placed on or in the pancreas and convey electrical signals responsive to pancreatic electric activity. Alternatively or additionally, one or more of electrodes 100 and any other electrodes coupled to control unit 90 may also serve as sense electrodes. Optionally, one or more supplemental sensors 72 (e.g., blood sugar, SvO2, pH, pCO2 or pO2 sensors) are coupled to convey data to the control unit, and are placed on or in the pancreas or elsewhere on or in the patient's body. Preferably, the control unit modifies the energy applied through electrodes 100 responsive to signals from sensors 72 and local sense electrodes 74, as described hereinbelow.

It is to be understood that the placement and number of electrodes and sensors in FIG. 1 are shown by way of example. Other sites on pancreas 20 or in a vicinity thereof are appropriate for electrode and sensor placement in other applications of the present invention. Different types of electrodes known in the art are typically selected based on the patient's specific medical condition, and may comprise substantially any suitable electrode known in the art of electrical stimulation or sensing in tissue.

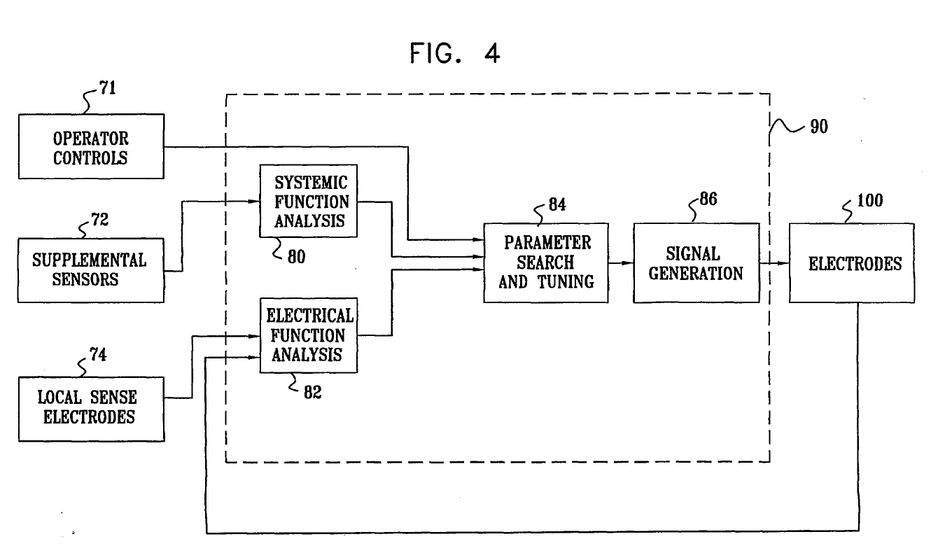

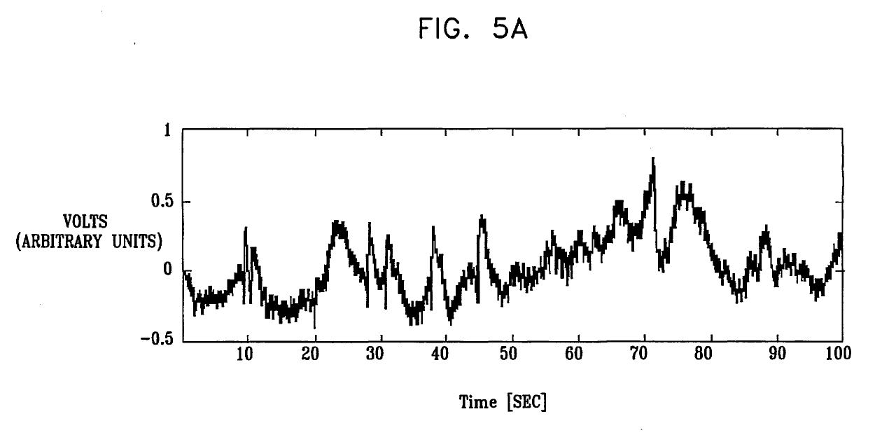

FIG. 4 is a schematic block diagram of control unit 90, in accordance with a preferred embodiment of the present invention. Local sense electrodes 74 and/or others of electrodes 100 are preferably coupled to provide feedback signals to an electrical function analysis block 82 of control unit 90. The feedback signals preferably provide information about various aspects of the pancreas' electrical activity to block 82, which analyzes the signals and, optionally, actuates control unit 90 to initiate or modify electrical energy applied to the pancreas responsive to the analysis. Alternatively or additionally, other responses to the measurements are implemented, such as the initiation or termination of insulin administration from an implanted pump. Preferably, signals applied to the pancreas are adjusted by the control unit, responsive to the feedback signals, in order to yield a desired response, e.g., a predetermined pancreatic electrical profile.

Preferably, block 82 conveys results of its analysis to a “parameter search and tuning” block 84 of control unit 90, which iteratively modifies characteristics of the electrical signals applied to the pancreas in order to attain a desired response. Further preferably, operating parameters of block 84 are entered during an initial calibration period by a human operator of the control unit using operator controls 71, which may comprise a keyboard or mouse. Block 84 typically utilizes multivariate optimization and control methods known in the art in order to cause one or more of the aforementioned electrical, chemical and/or other measured parameters to converge to desired values.

In general, each one of electrodes 100 may convey a particular waveform to pancreas 20, differing in certain aspects from the waveforms applied by the other electrodes. The particular waveform to be applied by each electrode is determined by control unit 90, initially under the control of the operator. Aspects of the waveforms which are set by the control unit, and may differ from electrode to electrode, typically include parameters such as time shifts between application of waveforms at different electrodes, waveform shapes, amplitudes, DC offsets, durations, and duty cycles. For example, the waveforms applied to some or all of electrodes 100 may comprise a monophasic square wave pulse, a sinusoid, a series of biphasic square waves, or a waveform including an exponentially-varying characteristic. Generally, the shape, magnitude, and timing of the waveforms are optimized for each patient and for each electrode, using suitable optimization algorithms as are known in the art. For example, one electrode may be driven to apply a signal, while a second electrode on the pancreas is not applying a signal. Subsequently, the electrodes may change functions, whereby the second electrode applies a signal, while the first electrode is not applying a signal.

For the purposes of this embodiment of the present invention, block 84 typically modifies a set of controllable parameters of the signals, responsive to the measured parameters, in accordance with values in a look-up table and/or pre-programmed formulae stored in an electronic memory of control unit 90. The controllable parameters may comprise, for example, pulse timing, magnitude, offset, and monophasic or biphasic shape. Preferably, the controllable parameters are conveyed by block 84 to a signal generation block 86 of control unit 90, which generates, responsive to the parameters, electrical signals that are applied by electrodes 100 to pancreas 20. Block 86 preferably comprises amplifiers, isolation units, and other standard circuitry known in the art of electrical signal generation.

In a typical initial calibration procedure, a bolus dose of glucose is administered to the patient, and electrical function analysis block 82 determines changes in the electrical activity of the pancreas responsive to the glucose. (Experimental results showing some such changes in activity are described hereinbelow.) Parameter search and tuning block 84 subsequently modifies a characteristic (e.g., timing, frequency, duration, magnitude, and/or shape) of the signals applied through one of electrodes 100, typically so as to cause the pancreas to release a hormone such as insulin in greater quantities than would otherwise be produced. This release causes cells throughout the patient's body to increase their uptake of the glucose, which, in turn, lowers the levels of glucose in the blood and causes the electrical activity of the pancreas to return to baseline values. In a series of similar calibration steps, block 84 repeatedly modifies characteristics of the signals applied through each of the electrodes, such that those modifications that reduce blood sugar, accelerate the return of the electropancreatographic measurements to baseline values, and/or otherwise improve the EPG, are generally maintained, while modifications that cause it to worsen are typically eliminated or avoided.

It will be appreciated that whereas the calibration procedure described hereinabove is applied with respect to a single electrode, for some applications, multiple electrodes are calibrated substantially simultaneously, for example, in order to determine which electrodes should be driven simultaneously to apply current to the pancreas.

Alternatively or additionally, the calibration procedure includes: (a) administration of insulin and/or a fasting period to reduce blood sugar levels, (b) detection of changes in pancreatic electrical activity responsive to the reduced blood sugar levels, and (c) application of electrical signals to the pancreas configured to enhance glucagon production and generally restore the EPG to its baseline.

Preferably, the calibration procedure is subsequently performed by a physician at intermittent follow-up visits and by unit 90 automatically during regular use of the apparatus (e.g., once per day, before and/or after a meal, or before and/or after physical activity), mutatis mutandis. When apparatus 18 is calibrated in the presence of a physician, it is often desirable to administer to the patient glucose boluses having a range of concentrations, in order to derive a broader range of operating parameters, which are stored in control unit 90 and can be accessed responsive to signals from the sensors and electrodes coupled to the control unit.

It is to be understood that preferred embodiments of the present invention are described herein with respect to glucose and insulin by way of example only. In other embodiments, the effects of other chemicals on pancreatic electrical activity are monitored, and/or signals are applied to the pancreas so as to modulate the release of other hormones. It is also to be understood that although electrodes 74 and 76 are shown for clarity of explanation as separate entities, a single set of electrodes may be used to perform both sensing and signal application.

Optionally, during the initial calibration procedure, the locations of one or more of electrodes 100 are varied while EPG signals are measured and/or electrical signals are applied therethrough, so as to determine optimum placement of the electrodes.

Preferably, during calibration and during regular operation of control unit 90, a systemic function analysis block 80 of control unit 90 receives inputs from supplemental sensors 72, and evaluates these inputs to detect an indication that blood sugar levels may be too high or too low. If appropriate, these inputs may be supplemented by user inputs entered through operator controls 71, indicating, for example, that the patient senses that her blood sugar is too low. In a preferred embodiment, parameter search and tuning block 84 utilizes the outputs of analysis blocks 80 and 82 in order to determine parameters of the signals which are applied through electrodes 100 to pancreas 20.

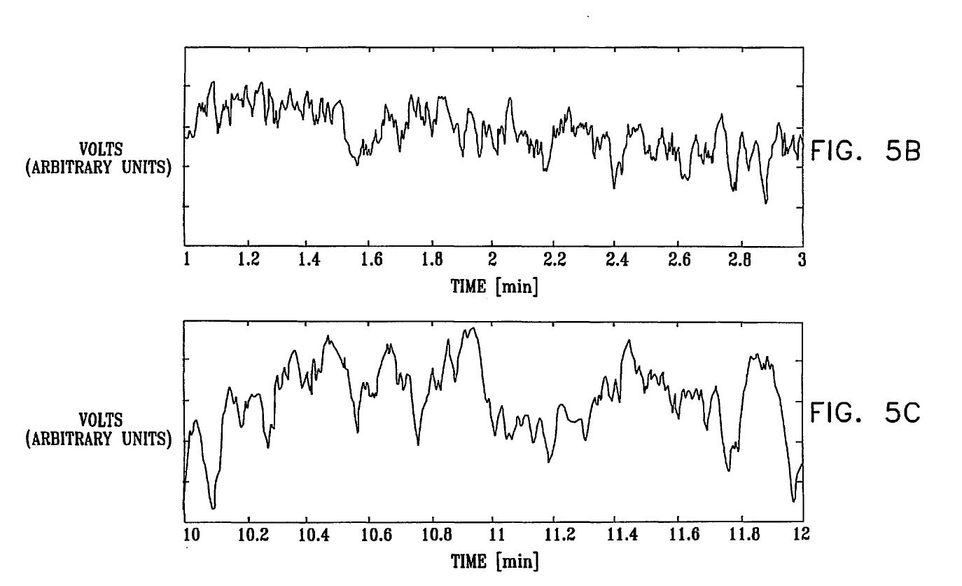

FIGS. 5A, 6A, 7B, and 7C are graphs showing in vivo experimental results measured in accordance with a preferred embodiment of the present invention. A sand rat (psammomys) was anesthetized with 40 mg/ml (0.15 mg/100 mg body weight) pentobarbital. The right jugular vein was cannulated to allow drug or glucose injections, and to allow blood samples to be taken for glucose concentration measurements. The animal was positioned on a warmed (37° C.) table. A laparotomy was performed, and the pancreas was displaced from the abdomen and put in a dish on top of an electrode set similar to that shown in FIG. 3B, while retaining anatomical connection to the rest of the body of the sand rat. By removing the pancreas from the body, breathing and ECG artifacts were reduced. Surface electrodes like those shown in FIG. 2 were carefully attached to the pancreas, and an additional set of electrodes like those shown in FIG. 3A were placed above the pancreas. The surgery and electrode placement were performed using surgical binoculars. In order to minimize electrical and mechanical noise, the sand rat was put inside a Faraday cage, and electrical measurements were performed on a pneumatic table.

The electrodes were connected to a Cyber-Amp 320 (Axon Instruments) amplifier, in which total gain was set to 10000 and a band pass filter was to allow 0.1 to 40 Hz signals to pass. The Cyber-Amp was connected to a computer, and recorded signals which were sampled at 1000 Hz and saved for off-line analysis.

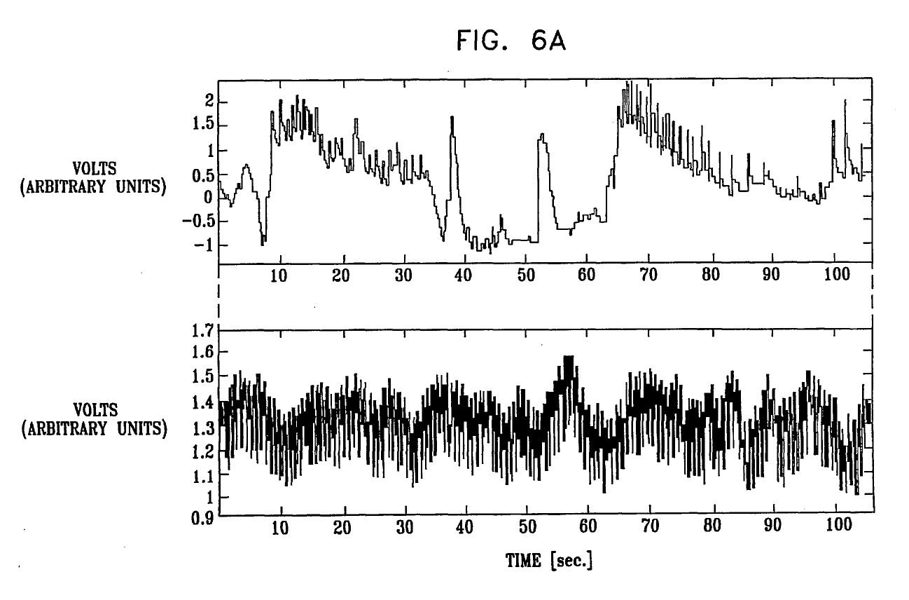

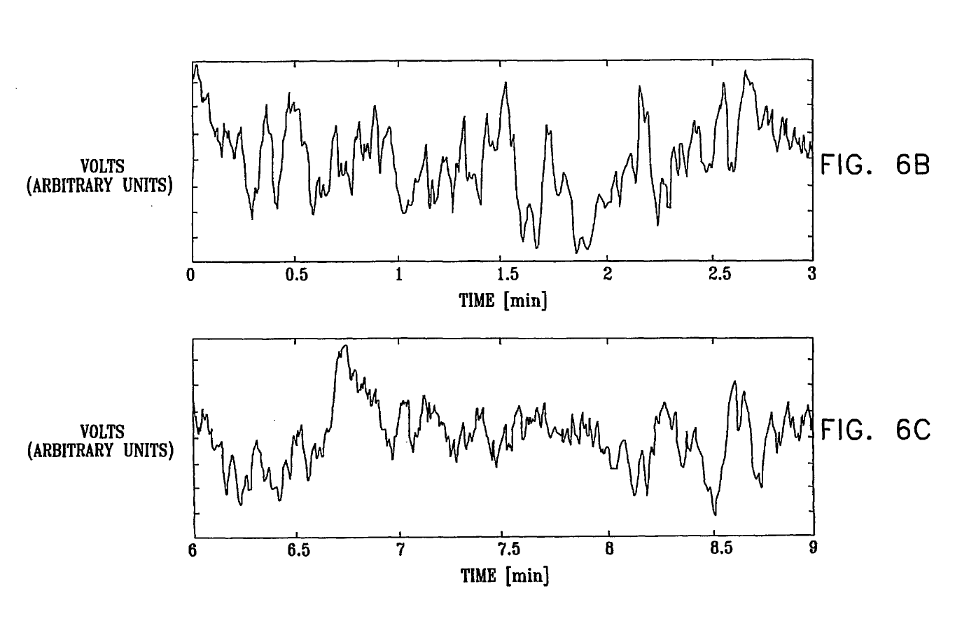

FIGS. 5A and 6A show bipolar pancreatic readings made at different times during experiments performed without the administration of glucose or any drug. It is noted that spikes of different widths are present in FIG. 5A, most being substantially longer, infrequent, and generally irregular than most of the spikes seen in FIG. 6A (e.g., those spikes generated at times t between 65 and 80 seconds). Much of the activity seen in FIG. 6A is characterized by sharply-rising spikes having durations between about 200 and 500 milliseconds, which are produced at a variable spike-generation rate having a mean value of about 1 Hz. The absolute amplitudes of the spikes are generally several tens of microvolts. As described in greater detail hereinbelow, waveform characteristics (such as spike widths) are preferably interpreted by a control unit to yield information about the activity of the various types of cells in the pancreas. For example, as shown in figures in the above-cited article by Nadal, beta cells typically produce spikes having widths which are markedly smaller than those of alpha cells. Alternatively or additionally, duration aspects and/or magnitude aspects of other features of the recorded waveform are analyzed to facilitate a determination by the control unit of the contribution of different types of pancreatic cells to the measured EPG.

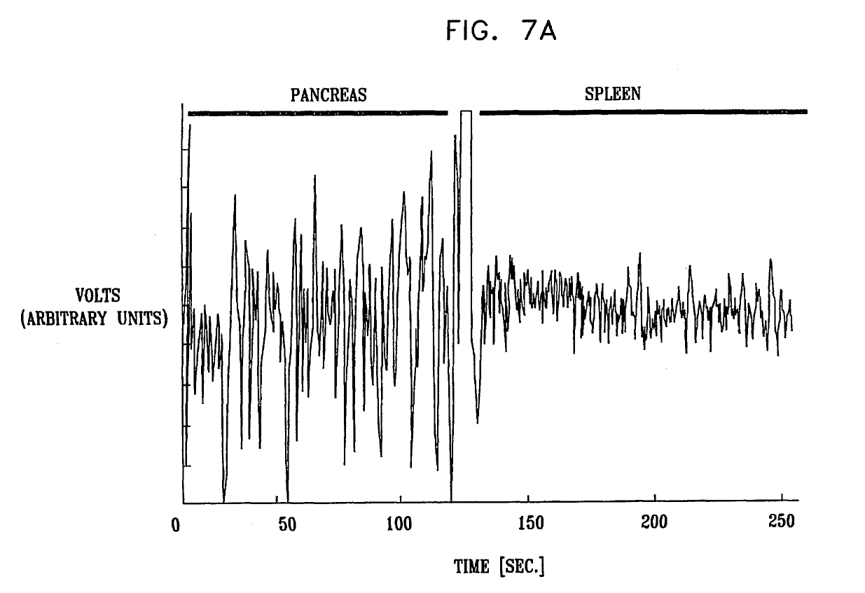

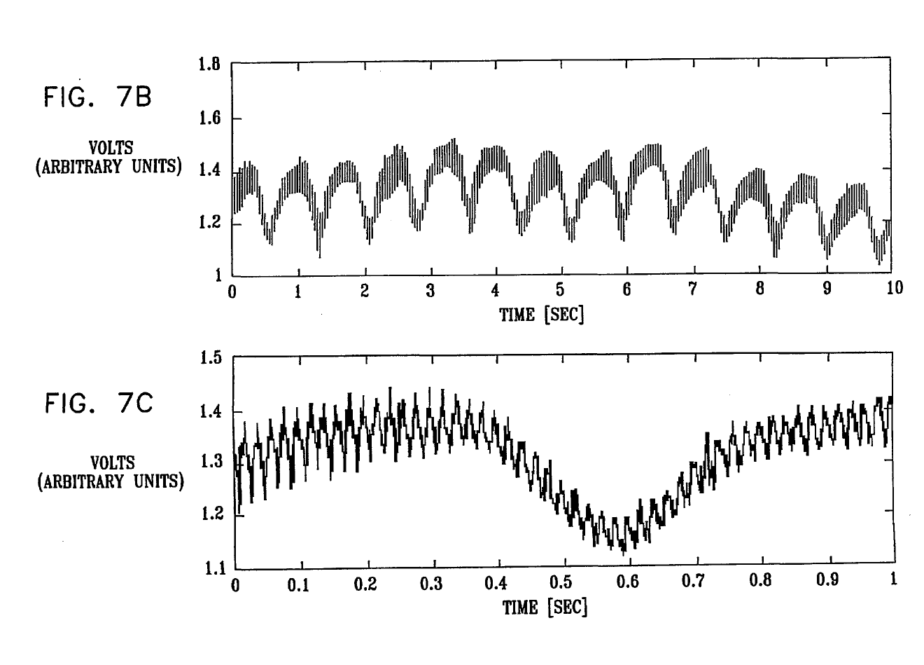

The lower trace in FIG. 6A shows noise measured by electrodes at a different site on the pancreas. To increase clarity, the time axis of this trace is expanded in FIG. 7B, and even further in FIG. 7C. The predominant features in FIG. 7B arise from breathing of the animal, while those in FIG. 7C are a result of power-line noise. It is noted that each of these is significantly different from the various pancreatic readings shown in the figures of the present patent application, and that software running in the control unit is preferably configured to identify and filter out any such non-pancreatic electrical activity.

FIGS. 5B, 5C, 6B, 6C, 7A, 8A, 8B, and 8C are graphs illustrating experimental data obtained in accordance with a preferred embodiment of the present invention. In these experiments, a rat was anesthetized, an abdominal incision was made in the animal, and the pancreas was removed from the rat's abdomen and placed in a Petri dish adjacent to the rat. Care was taken to assure that the major blood vessels connected to the pancreas were not cut or significantly disturbed during this procedure. The pancreas was removed so as to minimize the interference of the motion of breathing or other movements on the measurements being made. While in the Petri dish, the pancreas was continuously bathed in a warm saline solution.

Bipolar titanium wire electrodes, 300 microns in diameter, were placed in a mount similar to that shown in FIG. 2. The mount was placed on the head of the pancreas, in such a manner that the electrodes were sensitive to, it is believed, the electrical activity of at least several islets of Langerhans. In order to reduce electrical noise artifact, a sensing electrode was placed on the animal's spleen (in situ), which is substantially not electrically active. The data shown in FIGS. 5B, 5C, 6B, and 6C are voltage measurements reflecting the difference between the voltages measured on the pancreas and on the spleen.

The data in FIG. 5B represent a 2 minute baseline data collection period, in which the bipolar electrodes described hereinabove were held against the pancreas while data were recorded. Subsequently, a 20% glucose solution was injected into the rat. Pancreatic electrical activity subsequent to the injection is shown in FIG. 5C. A number of changes are seen between the baseline data and the post-injection data, including changes in frequency components of the recorded signal, as well as changes in magnitudes of fluctuations of the signal.

The data in FIG. 6B represent a 3 minute baseline data collection period, in which the bipolar electrodes were held against the pancreas while data were recorded. Subsequently, a 20% glucose solution was used to bathe the pancreas (rather than being injected into the rat). Pancreatic electrical activity subsequent to this administration of glucose is shown in FIG. 6C. As before, a number of changes are seen between the baseline data and the post-glucose-administration data, including changes in frequency components of the recorded signal, and changes in magnitudes of fluctuations of the signal.

It is believed that the types of changes shown in FIGS. 5B, 5C, 6B, and 6C, and/or other changes recognizable by a person skilled in the art of bioelectrical signal analysis, will be obtained in corresponding clinical applications of the technology described herein, mutatis mutandis. It is further believed that, following a suitable calibration period, this method of recording pancreatic electrical activity can be used in the detection of changes in glucose levels and/or the levels of other chemicals in the blood. Preferably, as described herein, these changes can be stored and used in a purely diagnostic fashion, or used in combination with therapeutic means, such as electrical signal application to the pancreas, or administration of insulin to the patient.

It is hypothesized that increases in amplitudes and/or fluctuations of the recorded signals may correspond to “recruitment” (activation) of increasing numbers of cells in increasing numbers of islets of Langerhans, which in turn corresponds to the propagation of glucose through the pancreas. It is noted that this propagation phenomenon is not detectable using techniques which are currently known in the art.

FIG. 7A shows the sensitivity of the measurement apparatus used in these rat experiments to the electrical activity of the pancreas and the spleen. The data shown in FIG. 7A represent electrical readings from the pancreas from t=0 to approximately t=120 seconds. Following this initial period, the electrodes were removed from the pancreas and placed on the spleen, and splenic electrical activity was recorded from t=about 140 to 250 seconds. The pancreas is seen to be significantly more electrically active than the spleen. In continuations of this experiment (not shown), each time the electrodes were moved from the pancreas to the spleen, the electrical activity was seen to decrease. Additionally, when the electrodes were moved back to the pancreas, activity increased. This graph indicates that the electrical activity measured by electrodes on the pancreas do, in fact, measure pancreatic electrical activity, and are not simply recording electric currents whose source is outside the pancreas. If the latter were the case, then similar activity would be expected to be seen on the spleen.

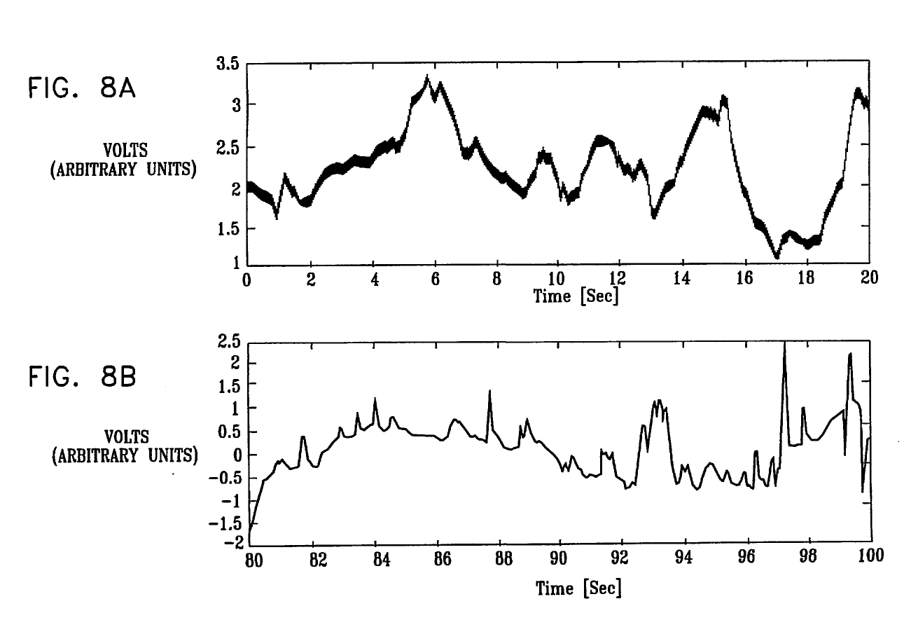

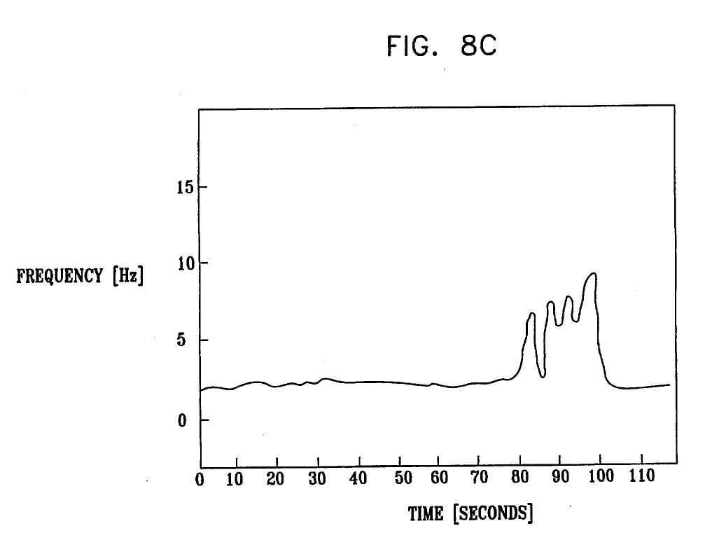

FIG. 8A shows electrical activity recorded in a sand rat during a first period (0-20 seconds). At approximately t=20 seconds, tolbutamide was injected. FIG. 8B shows pancreatic electrical activity during a second period (80-100 seconds), following this injection. It is noted that some frequency components are readily observable in FIG. 81B which are not present in FIG. 8A. FIG. 8C shows the results of a frequency analysis of all of the data, from 0 to 120 seconds. Dominant frequency components are clearly seen to change during the period following the injection of tolbutamide. In a preferred embodiment of the present invention, a control unit is adapted to analyze recorded electropancreatographic data so as to determine changes in the frequency components of the signal which are indicative of changes in a patient's blood sugar. For example, in the experiment whose results are shown in FIGS. 8A, 8B, and 8C, the effect of tolbutamide to increase pancreatic electrical activity, so as to stimulate insulin production and/or secretion, simulates the effect of high blood sugar to stimulate insulin production.

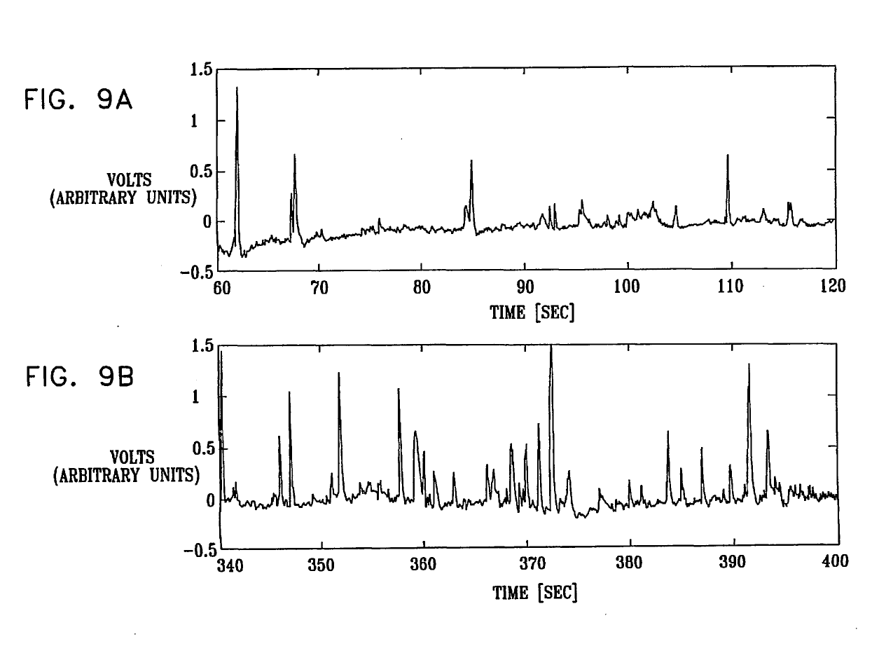

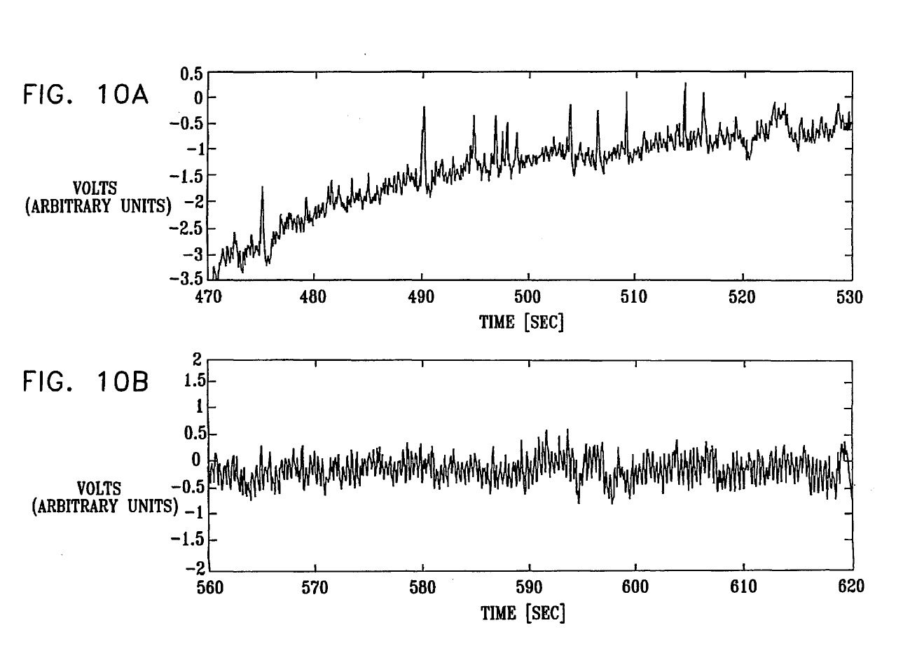

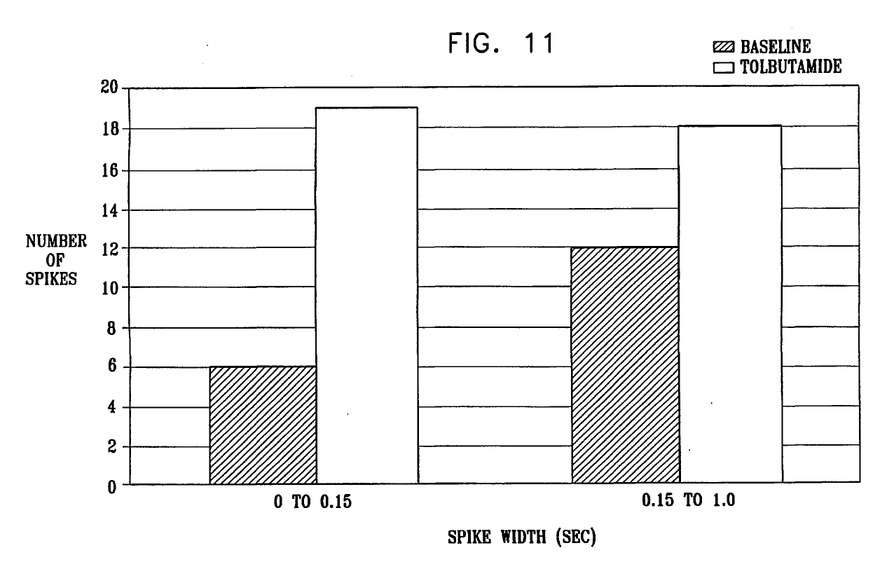

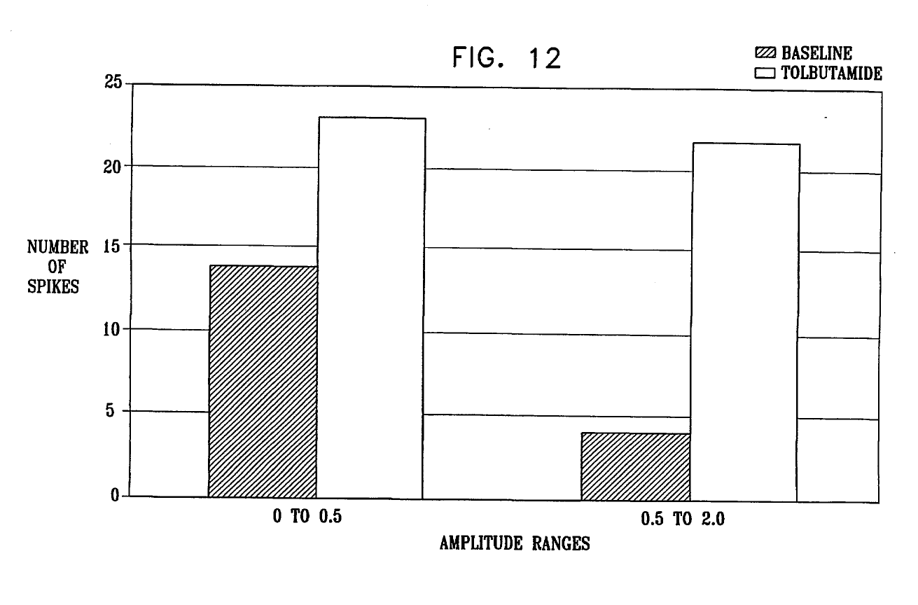

FIGS. 9A, 9B, 10A and 10B are graphs illustrating additional experimental data obtained in accordance with a preferred embodiment of the present invention. The experiments were performed upon sand rats under laboratory conditions similar to those of the experiments described above with reference to FIGS. 5B, 5C, 6B, 6C, 7A, 8A, 8B, and 8C. FIG. 9A shows a 2 minute baseline electrical activity data collection period, in which the bipolar electrodes on the pancreas recorded electrical activity. At approximately t=100 seconds, the sand rat was injected with a dose of tolbutamide (0.1 cc, 5 mM) through the jugular vein, in order to stimulate pancreatic electrical activity and thereby to increase the release of insulin. FIG. 9B shows data recorded through the same electrodes, beginning at four minutes after the tolbutamide injection. In FIG. 9B, a clear increase of electrical activity is observed in response to the administration of tolbutamide.

FIG. 10A shows a one minute baseline date collection period, in which the electrical activity of the pancreas of a sand rat was measured under similar laboratory conditions. At t=530 seconds, the sand rat was injected with diazoxide (0.1 cc), in order to reduce pancreatic electrical activity and thereby reduce the production and/or secretion of insulin. FIG. 10B, which shows data starting from thirty seconds following this injection, shows a marked decrease in pancreatic electrical activity. In particular, spike production is seen to be essentially terminated. The combined results of FIGS. 9A, 9B, 10A, and 10B show that electropancreatography, as provided by these embodiments of the present invention, can be used to allow a control unit implanted in a patient's body to determine in real-time whether the pancreas is behaving in a manner indicative of elevated blood sugar or depressed blood sugar. Responsive to such a determination, the control unit can, for example: (a) directly stimulate the pancreas so as to modulate insulin or glucagon production, (b) initiate other measures for restoring the pancreatic homeostasis, e.g., direct the patient to inject insulin or call for professional help, or (c) store recorded data to allow subsequent analysis.

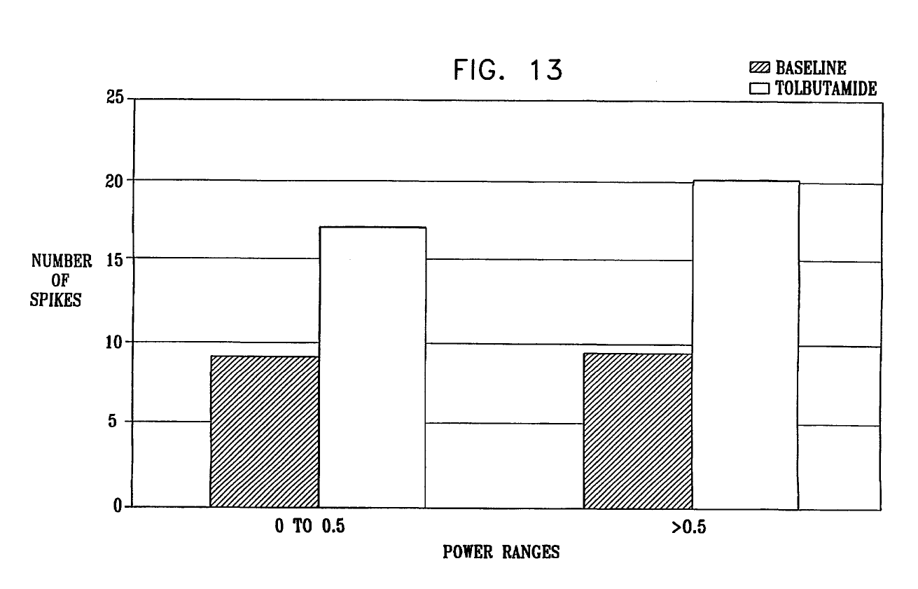

FIGS. 11, 12, and 13 show the results of signal processing of the experimental results shown in FIGS. 9A and 9B, in accordance with a preferred embodiment of the present invention. The width (duration) of each of the spikes measured during the experiment (of which the data shown in FIGS. 9A and 9B are a subset) was used as an indicator for dividing the spikes into two groups: Group I, those spikes having widths less than 0.15 second, and Group II, those spikes having widths ranging from 0.15 to 1.0 second. It can be seen in FIG. 11 that, for all ranges of measured spike width, the number of spikes after injection of tolbutamide is notably greater than prior to the tolbutamide injection. Thus, these data indicate that (under similar circumstances) a control unit which detects a similar increase in spike generation can attribute the increase to a systemic physiological change in the subject (e.g., changes in blood sugar).

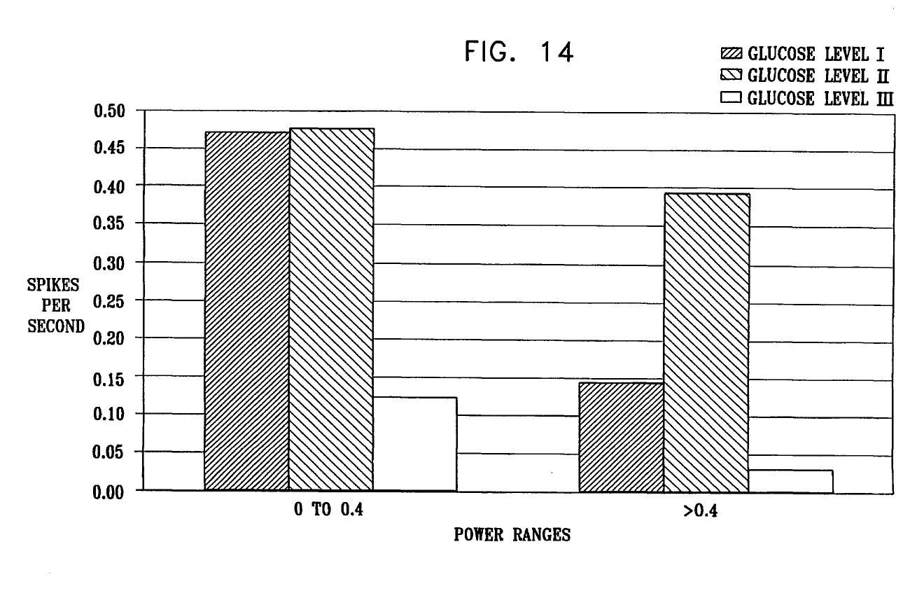

A similar analysis was performed with respect to the amplitudes of the spikes before and after tolbutamide injection. FIG. 12 shows that tolbutamide injection induces more large amplitude and small amplitude spikes than are present in the baseline state.

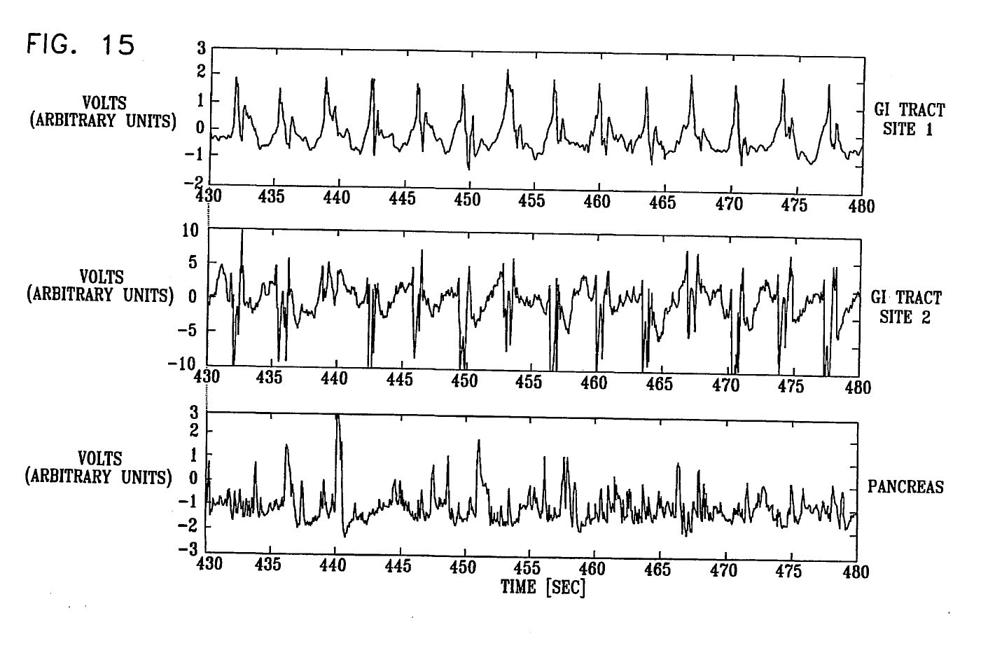

FIG. 13 is based on further analysis analogous to that shown in FIGS. 11 and 12. The width and the amplitude of each spike in FIGS. 9A and 9B were multiplied, so as to generate a measure of the power of the spike. It is seen that the injection of tolbutamide yields approximately twice the number of spikes relative to baseline, in the measured power ranges. These results indicate that electropancreatography, as provided by embodiments of the present invention, generates a quantitative indication of a condition of the blood. Thus, for example, this form of analysis can be used by a control unit implanted in a human to determine the onset and extent of glucose changes in the blood, mutatis mutandis.

FIG. 14 provides further support for this conclusion. In vivo, in situ, experiments were performed on the pancreas of a dog, in accordance with a preferred embodiment of the present invention. In these experiments, a portion of the outer layer of connective tissue surrounding the pancreas was removed, and surface electrodes were placed directly on the dog's pancreas. Results are shown in FIG. 14. In these experiments, three different levels of blood glucose were measured: Level I was approximately 170 mg/dL, Level II was approximately 220 mg/dL, and Level III was approximately 500 mg/dL. Electrical activity of the pancreas was measured responsive to each of the glucose levels. FIG. 14 shows the results of signal processing of the measured electrical activity similar to that described with reference to FIG. 13. It can be seen in FIG. 14 that the different glucose levels result in measurable differences in pancreatic electrical reaction. In particular, the excessively-high Level m protocol appears to either suppress spike generation, or not to facilitate it to the same extent as Levels I and II. In addition, glucose concentrations at Level II are seen to induce “high-power” spikes at over twice the rate of either Level I or Level III. Thus, FIG. 14 demonstrates that electropancreatography can be used to monitor the level of glucose in the blood. In clinical use, electropancreatographic readings would preferably be taken over a range of imposed glucose levels during calibration, so as to enable subsequent accurate assessments by the control unit of the patient's glucose levels.

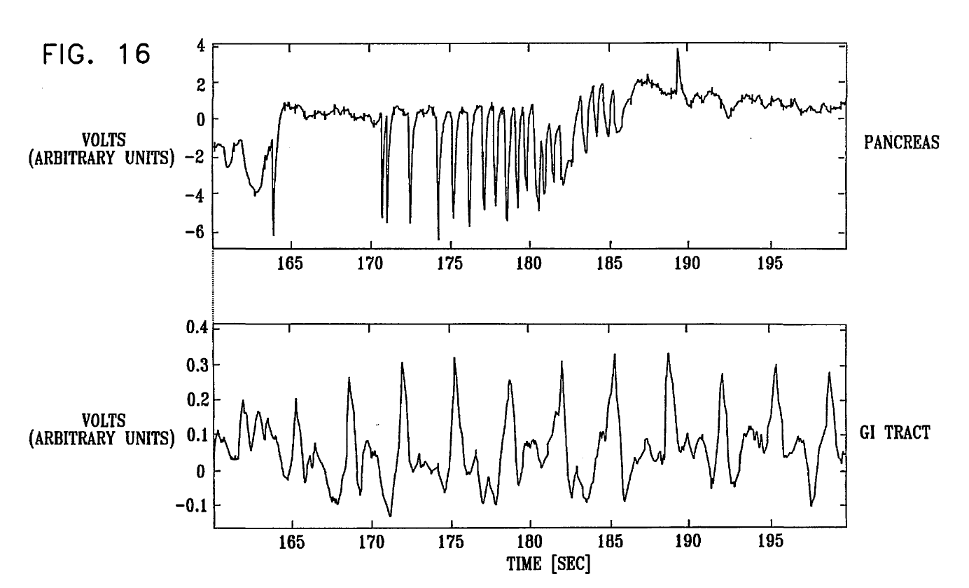

FIG. 15 shows results of a further experiment carried out in accordance with a preferred embodiment of the present invention. In order to ensure that the results of the above experiments and clinical electropancreatographic measurements do not include excessive electrical artifact due to electrical activity of smooth muscle in the vicinity of the pancreas, such as that of the gastrointestinal (GI) tract, measurements were made of the electrical activity at two sites in the GI tract simultaneous with the electropancreatographic measurements. The top and middle traces of FIG. 15 show the electrical activity at two sites on the GI tract of a dog, and the bottom trace shows the electrical activity of the pancreas, measured simultaneously with the GI tract measurements. It is markedly clear that the electrical activity of the GI tract is strongly periodic in nature, each GI site having the same period, while the pancreatic activity is independent of the GI tract. In the dog experiments described herein, a clip including a small metal spring was used to hold the electrode mounts to the pancreas.

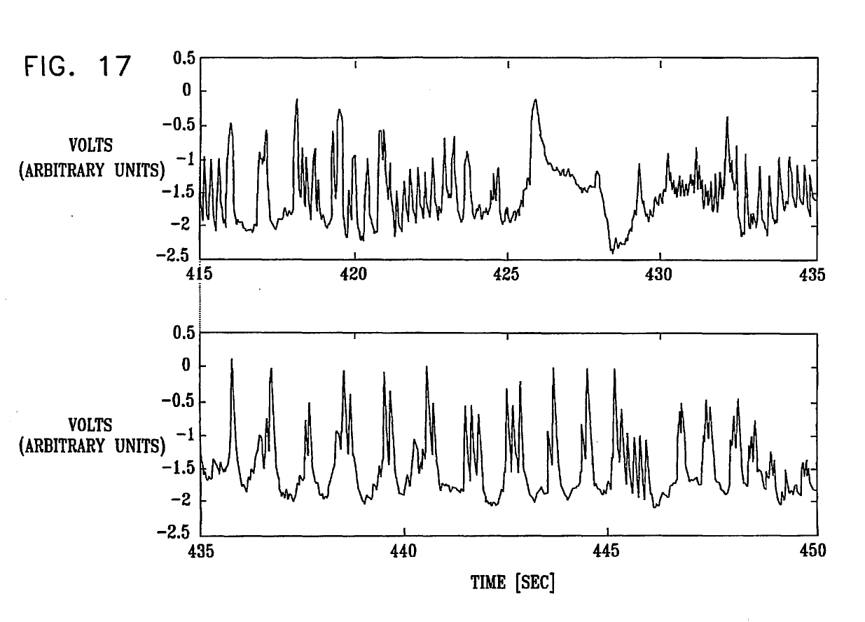

FIG. 16 shows results of yet a further experiment on a dog, comparing electropancreatographic readings with electrical activity measured at a site on the GI tract, in accordance with a preferred embodiment of the present invention. The electrical activity of the GI tract is distinctly periodic while the pancreas exhibits characteristic frequency changes. In particular, it is noted that the EPG trace shows a period of minimal pancreatic activity from t=165-170 seconds, which is followed by a ten second period in which spikes occur at continually increasing frequencies. This characteristic of the pancreas is both different from typical GI tract behavior, and has been seen by the inventors to recur in numerous experiments performed in accordance with preferred embodiments of the present invention. In clinical use, a control unit preferably monitors changes in the spike frequency responsive to a series of imposed or other conditions (such as particular glucose levels or changes in glucose levels), in order to determine those characteristic changes in spike frequency which are indicative that a treatment should be initiated or a warning signal should be generated. For example, in the calibration period for a given patient, any one or more of the following may be found to be useful indicators of blood glucose level or changes thereof:

a rate of spike generation,

aspects of the widths of one or more spikes,

aspects of morphology of a measured waveform,

changes (e.g., increases or decreases) in the rate of spike generation,

particular spike magnitudes associated with particular spike frequencies or with changes in spike frequencies,

changes in spike magnitudes associated with particular spike frequencies or with changes in spike frequencies, or

frequency or changes in frequency of spikes having particular spike widths, e.g., those widths which are predominantly characteristic of alpha-, beta-, delta-, or polypeptide-cell activity.

The GI tract data shown in FIGS. 15 and 16 are generally consistent with measurements of electrical activity of smooth muscles surrounding blood vessels made by several researchers and published in articles, such as those cited in the Background section of the present patent application by Lamb, F. S. et al., Zelcer, E., et al., Schobel, H. P., et al., and Johansson, B. et al.

FIG. 17 shows pancreatic electrical activity of a dog, measured in accordance with a preferred embodiment of the present invention. This data set is further indication that it is feasible to measure the electrical activity of a substantial portion of the pancreas and that the pattern of such activity is markedly different from the characteristic approximately 0.3 Hz electrical activity of the smooth muscle of the GI tract. Preferably, the effects of artifact due to various physiological factors such as smooth muscle electrical activity, neural activity, cardiac muscle activity and respiration, which are inherently distinguishable from pancreatic electrical activity because of their different characteristics, can be reduced in practice in preferred embodiments of the present invention, by: (a) the use of reference electrodes placed on or near a source of electrical artifact, or (b) software in the control unit which is operative to detect non-pancreatic waveforms and remove them from the EPG.

In a preferred mode of analysis, a control unit analyzes the EPG so as to distinguish between portions thereof which are indicative of activity of alpha cells and beta cells of the pancreas. For some applications, analysis is also performed to determine changes in delta cell activity and/or polypeptide cell activity. Increases in beta cell activity typically are interpreted by the control unit to be indicative of the generation of insulin responsive to increased blood sugar, while increases in alpha cell activity typically correspond to the generation of glucagon responsive to decreased blood sugar. If appropriate, a treatment may be initiated or modified based on these determinations.

Figures in the above-cited article by Nadal show calcium-based fluorescence changes responsive to alpha, beta, and delta cell activity. Each cell produces its own characteristic form, which distinguishes it from the other types of cells. A particular distinguishing characteristic is the duration of each burst of electrical activity. In the Nadal article, alpha cells are seen to produce substantially more prolonged, long-duration bursts of fluorescence than do beta cells, whose activity is better characterized as a series of short-duration spikes. The data presented in the figures of the present patent application can also be analyzed to distinguish between the activity of the different types of pancreatic cells. FIG. 17 shows prolonged, long-duration bursts of electrical activity, for example, between 425 and 428 seconds, at 417 seconds, and at 426 seconds, and repeated burst of short-duration spikes from 435 to 450 seconds. In a clinical setting, such an analysis is preferably performed following a suitable calibration of the EPG apparatus with each patient. The calibration preferably includes administering insulin or glucose in different doses to a patient to produce a range of blood sugar levels, and analyzing the EPG to determine characteristics of the spike associated with each blood sugar level.

For some applications, EPG analysis is performed using the assumption that the various inputs to the EPG (e.g., alpha-, beta-, delta-, and polypeptide-cells) are generally mutually independent. In this case, signal processing methods known in the art, such as single value decomposition (SVD) or principle component analysis, may be adapted for use in order to separate the overall recorded activity into its various sources.

Alternatively, for some applications it is preferred to assume that the various components of the EPG are mutually dependent, in which case techniques such as that described in the above-cited article by Gut are preferably adapted to enable a determination of the contribution to the EPG of alpha cells, beta cells, and/or other factors. In particular, the Gut article describes methods for distinguishing the contributions of individual finite-duration waveforms to an overall electromyographic (EMG) signal. In a preferred embodiment of the present invention, this method is adapted to facilitate a calculation of the contributions of groups of alpha and beta cells to the overall EPG signal.

It is to be understood that, in combination with or separately from the analysis methods described hereinabove, an EPG can be interpreted using simple methods, such as evaluating waveform frequencies, amplitudes, numbers of threshold-crossings, energy, correlations with predefined patterns or with an average pattern, or other characteristics.

It will be appreciated that the principles of the present invention can be embodied using a variety of types and configurations of hardware. For example, for some applications, it is appropriate to use a relatively small number of electrodes placed on or in the head and/or body and/or tail of the pancreas. Alternatively or additionally, a larger number of electrodes, e.g., more than ten, may be placed on the pancreas, preferably but not necessarily incorporated into flexible or stiff electrode arrays. In a preferred embodiment, several arrays each comprising about 30-60 electrodes are placed on or implanted in the pancreas.

It is noted that the pin electrodes used in gathering the data shown in the figures had characteristic diameters of approximately 500 to 1000 microns, which, despite their large size, were able to record electrical activity over relatively long periods, e.g., up to several hours. Any injury which may have been induced (none was detected) would presumably have been limited to a local region around each electrode. For some clinical applications, it is preferable to use or adapt for use commercially-available electrodes such as those which have diameters of several microns and are designed for recording electrical activity in the brain. A range of electrodes are known or could be adapted to measure the characteristic 1-100 microvolt pancreatic electrical activity

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art which would occur to persons skilled in the art upon reading the foregoing description.

1. Apparatus for sensing electrical activity of a pancreas of a patient, comprising:
a set of one or more electrodes, adapted to be coupled to the pancreas; and
a control unit, adapted to receive electrical signals from the electrodes indicative of electrical activity of pancreatic cells which are in a plurality of islets of the pancreas, and to generate an output responsive thereto.

2. Apparatus according to claim 1, wherein a single electrode in the set of one or more electrodes is adapted to convey to the control unit an electrical signal indicative of electrical activity of pancreatic cells which are in two or more of the islets.

3. Apparatus according to claim 1, wherein the control unit is adapted to receive electrical signals from the electrodes indicative of electrical activity of pancreatic cells which are in five or more of the islets.

4. Apparatus according to claim 1, wherein the control unit is adapted to receive electrical signals from the electrodes indicative of electrical activity of pancreatic cells which are in ten or more of the islets.

5. Apparatus according to claim 1, wherein a first one of the one or more electrodes is adapted to convey to the control unit a first electrical signal, indicative of electrical activity of pancreatic cells which are in a first one of the islets, and wherein a second one of the one or more electrodes is adapted to convey to the control unit a second electrical signal, indicative of electrical activity of pancreatic cells which are in a second one of the islets, which is different from the first one of the islets.

6. Apparatus according to claim 1, wherein the control unit is adapted to receive the electrical signals from the electrodes responsive to spontaneous electrical activity of the pancreatic cells.

7. Apparatus according to claim 1, wherein the control unit is adapted to analyze the signals so as to identify an aspect thereof indicative of activity of a type of cell selected from the list consisting of: pancreatic alpha cells, pancreatic beta cells, pancreatic delta cells, and polypeptide cells, and wherein the control unit is adapted to generate the output responsive to identifying the aspect.

8. Apparatus for monitoring a glucose level of a patient, comprising:
a set of one or more electrodes, adapted to be coupled to a pancreas of the patient; and
a control unit, adapted to receive electrical signals from the electrodes indicative of spontaneous electrical activity of pancreatic cells, to analyze the signals so as to determine a change in the glucose level, and to generate an output responsive to determining the change.

9. Apparatus according to claim 8, wherein the control unit is adapted to analyze the signals so as to identify an aspect thereof indicative of activity of a type of cell selected from the list consisting of: pancreatic alpha cells, pancreatic beta cells, pancreatic delta cells, and polypeptide cells, and wherein the control unit is adapted to generate the output responsive to identifying the aspect.

10. Apparatus for analyzing electrical activity of a pancreas of a patient, comprising:
a set of one or more electrodes, adapted to be coupled to the pancreas; and
a control unit, adapted to receive electrical signals from the electrodes, adapted to analyze the signals so as to identify an aspect thereof which is indicative of activity of pancreatic alpha cells, and adapted to generate an output responsive to identifying the aspect.

11. Apparatus for analyzing electrical activity of a pancreas of a patient, comprising:
a set of one or more electrodes, adapted to be coupled to the pancreas; and
a control unit, adapted to receive electrical signals from the electrodes, adapted to analyze the signals so as to identify an aspect thereof which is indicative of spontaneous activity of pancreatic beta cells, and adapted to generate an output responsive to identifying the aspect.

12. Apparatus according to claim 11, wherein the control unit is adapted to analyze the signals so as to distinguish between the aspect thereof which is indicative of the activity of the beta cells and an aspect thereof which is indicative of activity of pancreatic alpha cells, and wherein the control unit is adapted to generate the output responsive to distinguishing between the aspects.

13. Apparatus for analyzing electrical activity of a pancreas of a patient, comprising:
a set of one or more electrodes, adapted to be coupled to the pancreas; and
a control unit, adapted to receive electrical signals from the electrodes, adapted to analyze the signals so as to identify an aspect thereof which is indicative of activity of pancreatic delta cells, and adapted to generate an output responsive to identifying the aspect.

14. Apparatus for analyzing electrical activity of a pancreas of a patient, comprising:
a set of one or more electrodes, adapted to be coupled to the pancreas; and
a control unit, adapted to receive electrical signals from the electrodes, adapted to analyze the signals so as to identify an aspect thereof which is indicative of activity of polypeptide cells, and adapted to generate an output responsive to identifying the aspect.

15. Apparatus according to any one of claims 10, 11, 13, or 14, wherein the control unit is adapted to compare the aspect of the signals with a stored pattern that is indicative of activity of the cells, and to generate the output responsive thereto.

16. Apparatus according to any one of claims 10, 11, 13, or 14, wherein the control unit is adapted to analyze the signals by means of a technique selected from the list consisting of: single value decomposition and principle component analysis, and to generate the output responsive thereto.

17. Apparatus according to any one of claims 10, 11, 13, or 14, wherein the control unit is adapted to analyze the signals under an assumption that the activity of the cells is dependent on electrical activity of another type of pancreatic cell, and to generate the output responsive thereto.

18. Apparatus according to any one of claims 10, 11, 13, or 14, wherein the control unit is adapted to analyze the signals so as to identify a frequency aspect thereof, and to generate the output responsive to identifying the frequency aspect.

19. Apparatus according to claim 18, wherein the control unit is adapted to analyze the signals so as to differentiate between a first frequency aspect of the signals which is indicative of the activity of the cells, and a second frequency aspect of the signals, different from the first frequency aspect, which is indicative of activity of another type of pancreatic cell.

20. Apparatus according to claim 18, wherein the control unit is adapted to analyze the signals so as to identify over time a change in the frequency aspect that is characteristic of the cells.

21. Apparatus according to claim 18, wherein the control unit is adapted to analyze the signals so as to identify a magnitude aspect thereof, wherein the control unit is adapted to analyze the frequency aspect and the magnitude aspect in combination, and wherein the control unit is adapted to generate the output responsive to analyzing the aspects.

22. Apparatus according to claim 18, wherein the control unit is adapted to analyze the signals so as to identify a duration aspect thereof, wherein the control unit is adapted to analyze the frequency aspect and the duration aspect in combination, and wherein the control unit is adapted to generate the output responsive to analyzing the aspects.

23. Apparatus according to any one of claims 1, 8, 10, 11, 13, or 14, wherein the control unit is adapted to analyze the signals so as to identify a magnitude aspect thereof, and to generate the output responsive to identifying the magnitude aspect.

24. Apparatus according to any one of claims 1, 8, 10, 11, 13, or 14, wherein the control unit is adapted to analyze the signals so as to identify a duration aspect thereof, and to generate the output responsive to identifying the duration aspect.

25. Apparatus according to any one of claims 1, 8, 10, 11, 13, or 14, wherein the control unit is adapted to analyze the signals so as to identify a magnitude aspect thereof and a duration aspect thereof, wherein the control unit is adapted to analyze the aspects in combination, and wherein the control unit is adapted to generate the output responsive to analyzing the aspects.

26. Apparatus according to any one of claims 1, 8, 10, 11, 13, or 14, wherein the control unit is adapted to analyze the electrical signals with respect to calibration data indicative of aspects of pancreatic electrical activity recorded at respective times, in which respective measurements of blood glucose level of the patient generated respective values.

27. Apparatus according to any one of claims 1, 8, 10, 11, 13, or 14, wherein the electrodes are adapted to be placed in contact with the pancreas.

28. Apparatus according to claim 27, wherein at least one of the electrodes is adapted to be placed in contact with the head of the pancreas.

29. Apparatus according to claim 27, wherein at least one of the electrodes is adapted to be placed in contact with the body of the pancreas.

30. Apparatus according to claim 27, wherein at least one of the electrodes is adapted to be placed in contact with the tail of the pancreas.

31. Apparatus according to claim 27, wherein at least one of the electrodes is adapted to be placed in contact with a vein or artery of the pancreas.

32. Apparatus according to any one of claims 1, 8, 10, 11, 13, or 14, wherein at least one of the electrodes is adapted to be placed in contact with a blood vessel in a vicinity of the pancreas.

33. Apparatus according to any one of claims 1, 8, 10, 11, 13, or 14, wherein the apparatus comprises a treatment unit, adapted to receive the output and to apply a treatment to the patient responsive to receiving the output.

34. Apparatus according to claim 33, wherein the treatment unit comprises at least one electrode in the set of electrodes, and wherein the control unit is adapted to drive the at least one electrode to apply current to the pancreas capable of treating a condition of the patient.

35. Apparatus according to claim 33, wherein the treatment unit comprises a signal-application electrode, and wherein the control unit is adapted to drive the signal-application electrode to apply current to the pancreas capable of treating a condition of the patient.

36. Apparatus according to claim 33, wherein the control unit is adapted to generate the output responsive to an aspect of the timing of the electrical signals, and wherein the treatment unit is adapted to apply the treatment responsive to the timing aspect.

37. Apparatus according to claim 33, wherein the control unit is adapted to configure the output to the treatment unit so as to be capable of modifying an amount of glucose in blood in the patient.

38. Apparatus according to claim 37, wherein the control unit is adapted to configure the output to the treatment unit so as to be capable of increasing an amount of glucose in blood in the patient.

39. Apparatus according to claim 37, wherein the control unit is adapted to configure the output so as to be capable of decreasing an amount of glucose in blood in the patient.

40. Apparatus according to any one of claims 1, 8, 10, 11, 13, or 14, wherein the control unit is adapted to receive the signals from at least one of the electrodes when the at least one of the electrodes is not in contact with any islet of the pancreas.

41. Apparatus according to any one of claims 1, 8, 10, 11, 13, or 14, wherein the control unit is adapted to generate the output so as to facilitate an evaluation of a state of the patient.

42. A method for sensing electrical activity of a pancreas of a patient, comprising:
receiving electrical signals indicative of electrical activity of pancreatic cells which are in a plurality of islets of the pancreas; and
generating an output responsive thereto.

43. A method according to claim 42, wherein receiving the electrical signals comprises receiving an electrical signal recorded at a single site of the pancreas, which signal is indicative of electrical activity of pancreatic cells which are in two or more of the islets.

44. A method according to claim 42, wherein receiving the electrical signals comprises receiving signals indicative of electrical activity of pancreatic cells which are in five or more of the islets.

45. A method according to claim 42, wherein receiving the electrical signals comprises receiving signals indicative of electrical activity of pancreatic cells which are in ten or more of the islets.

46. A method according to claim 42, wherein receiving the electrical signals comprises:
receiving a first electrical signal recorded at a first site, indicative of electrical activity of pancreatic cells which are in a first one of the islets; and
receiving a second electrical signal recorded at a second site, indicative of electrical activity of pancreatic cells which are in a second one of the islets, which is different from the first one of the islets.

47. A method according to claim 42, wherein receiving the electrical signals comprises receiving electrical signals responsive to spontaneous electrical activity of the pancreatic cells.

48. A method according to claim 42, and comprising analyzing the signals so as to identify an aspect thereof indicative of activity of a type of cell selected from the list consisting of: pancreatic alpha cells, pancreatic beta cells, pancreatic delta cells, and polypeptide cells, wherein generating the output comprises generating the output responsive to identifying the aspect.

49. A method for monitoring a glucose level of a patient, comprising:
receiving electrical signals indicative of spontaneous electrical activity of pancreatic cells;
analyzing the signals so as to determine a change in the glucose level; and
generating an output responsive to determining the change.

50. A method according to claim 49, wherein analyzing the signals comprises identifying an aspect thereof indicative of activity of a type of cell selected from the list consisting of: pancreatic alpha cells, pancreatic beta cells, pancreatic delta cells, and polypeptide cells, and wherein generating the output comprises generating the output responsive to identifying the aspect.

51. A method for analyzing electrical activity of a pancreas of a patient, comprising:
receiving electrical signals recorded at one or more pancreatic sites;
analyzing the signals so as to identify an aspect thereof which is indicative of activity of pancreatic alpha cells; and
generating an output responsive to identifying the aspect.

52. A method for analyzing electrical activity of a pancreas of a patient, comprising:
receiving electrical signals recorded at one or more pancreatic sites;
analyzing the signals so as to identify an aspect thereof which is indicative of activity of pancreatic beta cells; and
generating an output responsive to identifying the aspect.

53. A method according to claim 52, wherein analyzing the signals comprises distinguishing between the aspect thereof which is indicative of the activity of the beta cells and an aspect thereof which is indicative of activity of pancreatic alpha cells, wherein generating the output comprises generating the output responsive to distinguishing between the aspects.

54. A method for analyzing electrical activity of a pancreas of a patient, comprising:
receiving electrical signals recorded at one or more pancreatic sites;
analyzing the signals so as to identify an aspect thereof which is indicative of activity of pancreatic delta cells; and
generating an output responsive to identifying the aspect.

55. A method for analyzing electrical activity of a pancreas of a patient, comprising:
receiving electrical signals recorded at one or more pancreatic sites;
analyzing the signals so as to identify an aspect thereof which is indicative of activity of polypeptide cells; and
generating an output responsive to identifying the aspect.

56. A method according to any one of claims 51, 52, 54, or 55, wherein analyzing the signals comprises comparing the aspect of the signals with a stored pattern that is indicative of activity of the cells, and wherein generating the output comprises generating the output responsive thereto.

57. A method according to any one of claims 51, 52, 54, or 55, wherein analyzing the signals comprises analyzing the signals by means of a technique selected from the list consisting of: single value decomposition and principle component analysis, and wherein generating the output comprises generating the output responsive thereto.

58. A method according to any one of claims 51, 52, 54, or 55, wherein analyzing the signals comprises analyzing the signals under an assumption that the activity of the cells is dependent on electrical activity of another type of pancreatic cell, and wherein generating the output comprises generating the output responsive thereto.

59. A method according to any one of claims 51, 52, 54, or 55, wherein analyzing the signals comprises analyzing the signals so as to identify a frequency aspect thereof, and wherein generating the output comprises generating the output responsive to identifying the frequency aspect.

60. A method according to claim 59, wherein analyzing the signals comprises analyzing the signals so as to differentiate between a first frequency aspect of the signals which is indicative of the activity of the cells, and a second frequency aspect of the signals, different from the first frequency aspect, which is indicative of activity of another type of pancreatic cell.

61. A method according to claim 59, wherein analyzing the signals comprises analyzing the signals so as to identify over time a change in the frequency aspect that is characteristic of the cells.

62. A method according to claim 59, wherein analyzing the signals comprises:
analyzing the signals so as to identify a magnitude aspect thereof; and
analyzing the frequency aspect and the magnitude aspect in combination,
wherein generating the output comprises generating the output responsive to analyzing the aspects.

63. A method according to claim 59, wherein analyzing the signals comprises:
analyzing the signals so as to identify a duration aspect thereof; and
analyzing the frequency aspect and the duration aspect in combination,
wherein generating the output comprises generating the output responsive to analyzing the aspects.

64. A method according to any one of claims 42, 49, 51, 52, 54, or 55, wherein analyzing the signals comprises analyzing the signals so as to identify a magnitude aspect thereof, and wherein generating the output comprises generating the output responsive to identifying the magnitude aspect.

65. A method according to any one of claims 42, 49, 51, 52, 54, or 55, wherein analyzing the signals comprises analyzing the signals so as to identify a duration aspect thereof, and wherein generating the output comprises generating the output responsive to identifying the duration aspect.

66. A method according to any one of claims 42, 49, 51, 52, 54, or 55, wherein analyzing the signals comprises:
analyzing the signals so as to identify a magnitude aspect thereof and a duration aspect thereof; and
analyzing the aspects in combination,
wherein generating the output comprises generating the output responsive to analyzing the aspects.

67. A method according to any one of claims 42, 49, 51, 52, 54, or 55, wherein analyzing the signals comprises analyzing the electrical signals with respect to calibration data indicative of aspects of pancreatic electrical activity recorded at respective times, in which respective measurements of blood glucose level of the patient generated respective values.

68. A method according to any one of claims 42, 49, 51, 52, 54, or 55, wherein receiving the electrical signals comprises receiving the signals from at least one electrode placed in contact with the pancreas.

69. A method according to claim 68, wherein receiving the electrical signals comprises receiving the signals from at least one electrode placed in contact with the head of the pancreas.

70. A method according to claim 68, wherein receiving the electrical signals comprises receiving the signals from at least one electrode placed in contact with the body of the pancreas.

71. A method according to claim 68, wherein receiving the electrical signals comprises receiving the signals from at least one electrode placed in contact with the tail of the pancreas.

72. A method according to claim 68, wherein receiving the electrical signals comprises receiving the signals from at least one electrode placed in contact with a vein or artery of the pancreas.

73. A method according to any one of claims 42, 49, 51, 52, 54, or 55, wherein at least one of the electrodes is adapted to be placed in contact with a blood vessel in a vicinity of the pancreas.

74. A method according to any one of claims 42, 49, 51, 52, 54, or 55, and comprising applying a treatment to the patient responsive to receiving the output.

75. A method according to claim 74, wherein applying the treatment comprises applying electric current to the pancreas capable of treating a condition of the patient.

76. A method according to claim 74, wherein applying the treatment comprises applying the treatment responsive to an aspect of the timing of the electrical signals.

77. A method according to claim 74, wherein applying the treatment comprises configuring the treatment so as to be capable of modifying an amount of glucose in blood in the patient.

78. A method according to claim 77, wherein configuring the treatment comprises configuring the treatment so as to be capable of increasing an amount of glucose in blood in the patient.

79. A method according to claim 77, wherein configuring the treatment comprises configuring the treatment so as to be capable of decreasing an amount of glucose in blood in the patient.

80. A method according to any one of claims 42, 49, 51, 52, 54, or 55, wherein receiving the signals comprises receiving the signals from at least one electrode which is not in contact with any islet of the pancreas.

81. A method according to any one of claims 42, 49, 51, 52, 54, or 55, wherein generating the output comprises facilitating an evaluation of a state of the patient.

82. Apparatus according to any one of claims 1, 8, 10, 11, 13, or 14, wherein at least one of the electrodes has a characteristic diameter less than about 3 millimeters.

83. Apparatus according to claim 82, wherein the at least one of the electrodes has a characteristic diameter less than about 300 microns.

84. Apparatus according to claim 83, wherein the at least one of the electrodes has a characteristic diameter less than about 30 microns.

85. Apparatus according to any one of claims 1, 8, 10, 11, 13, or 14, and comprising a clip mount, coupled to at least one of the electrodes, which is adapted for securing the at least one of the electrodes to the pancreas.
